(19) United States
(12) Patent Application Publication
(10) Pub. No.: US 2023/0005618 A1
(43) Pub. Date: Jan. 5, 2023

(54) METHODS, APPARATUS, AND SYSTEMS FOR IMPROVING THE QUALITY OF PATIENT CARE

(71) Applicant: MacuLogix, Inc., Harrisburg, PA (US)

(72) Inventor: Gregory R. Jackson, Hershey, PA (US)

(21) Appl. No.: 17/792,541

(22) PCT Filed: Jan. 13, 2021
(86) PCT No.: PCT/US2021/013291

§ 371 Date: Jul. 13, 2022

Related U.S. Application Data
(60) Provisional application No. 62/960,643, filed on Jan. 13, 2020.

(51) Int. Cl. G16H 50/20; G16H 10/60; G16H 40/67

(52) U.S. Cl. CPC G16H 50/20; G16H 10/60; G16H 40/67

(57) ABSTRACT

Methods, apparatus, and systems for improving quality of patient care are described. the system can include an interactive interface that is configured to receive, from a practitioner or any skilled person, input for use in classifying the practitioners' patients into one or more groups based on certain identifying features. The identifying features can be any suitable features, such as the patient's age, medical history, family history, presence of a new medical condition (e.g., detection of possible diabetes during an ophthalmic test), etc. The system can also be configured to receive information from various patient databases, such as various electronic patient systems and electronic billing systems.

### RELATED APPLICATIONS

The Application claims the benefit of and priority to U.S. Provisional Application No. 62/960,643, filed on Jan. 13, 2020, the entire teachings of which is incorporated herein by reference.

### FIELD

The present disclosure generally relates to methods and systems for improving quality of patient care, and more particularly to methods, apparatus, and systems assisting or aiding a practitioner in providing uniform and improved medical care at a medical practice.

### BACKGROUND

Providing consistent and uniform health care to patients visiting a practice can be challenging. Often times, a physician can encounter multiple patients having various issues during a given day and, as such, he/she cannot always remember to provide their patient with consistent and effective care. For example, a practitioner can simply forget to administer a certain test to his/her patient, overlook a symptom or an important fact in the patient's medical history, and/or forget to provide the patient with effective continuity of care (e.g., follow up visits, specialist referrals, etc.).

These difficulties can also result in non-uniformity of the quality of patient care offered to patients within a medical practice. For example, while one practitioner in a medical practice may remember to check his/her patients' medical records to determine whether a specific patient has one or more markers for macular degeneration, and test that patient for presence of macular degeneration, that practitioner's colleagues, working within that same medical practice, may simply forget to check their patients' medical records for possible markers of macular degeneration and/or offer appropriate tests to their patients.

### SUMMARY

In one aspect, a system for improving quality of healthcare service provided to a patient of a medical practice is described. The system can include one or more processors, a memory coupled to the one or more processors, and one or more programs stored in the memory and configured to be executed by the one or more processors. The one or more programs can include instructions that upon execution: receive information identifying the patient of the medical practice; classify the patient into one or more categories based on at least one predetermined criterion; recommend at least one healthcare service action for providing to the patient; and provide a practitioner at the medical practice with information relating to the at least one healthcare service action.

In other examples, the aspects above, or any system, method, apparatus described herein can include one or more of the following features.

The information identifying the patient can comprise information stored on at least one electronic health record system that stores at least some information about the patient. Additionally or alternatively, the information identifying the patient can comprise information stored on an internal database of the medical practice. The information identifying the patient can further comprise information relating to at least one of patient's health records, patient's insurance records, patient's biographic records, patient's demographic records, and patient's insurance billing records.

The system can further comprise an interactive practitioner interface that is coupled to the processor and configured to provide the practitioner with the at least one healthcare service action. The one or more programs can further include instructions that upon execution receive the at least one criterion from the practitioner via the interactive practitioner interface. The at least one criterion can be at least one of a feature, a symptom, a condition, a medical history item, or a personal history item common to patients requiring the at least one healthcare service action. Further, the one or more programs include instructions that upon execution obtain information relating to the at least one criterion from the information identifying the patient. For example, if the at least one criterion is the age of the patient, the one or more programs can obtain this information from the patient's demographic records.

Further, the at least one healthcare service action can comprise a follow up action with the patient. Specifically, the system can comprise an interactive patient interface coupled to the processor and configured to receive at least some of information identifying the patient from the patient. The interactive patient interface that is coupled to the processor can further be configured to provide the patient with at least some information relating to the at least one healthcare service item. Additionally or alternatively, the interactive patient interface can further be configured to receive a response from the patient in connection with the at least one healthcare service item.

The one or more programs can further include instructions that upon execution analyze the response received from the patient and classify the response into the one or more updated categories based on the at least one predetermined criterion. Further, the one or more programs can include instructions that upon execution recommend at least one updated healthcare service action based on the one or more updated categories.

Other aspects and advantages of the invention can become apparent from the following drawings and description, all of which illustrate the various aspects of the invention, by way of example only.

### BRIEF DESCRIPTION OF THE DRAWINGS

A detailed description of various embodiments is provided herein below with reference, by way of example, to the following drawings. It will be understood that the drawings are exemplary only and that all reference to the drawings is made for the purpose of illustration only, and is not intended to limit the scope of the embodiments described herein below in any way. For convenience, reference numerals may also be repeated (with or without an offset) throughout the figures to indicate analogous components or features.

FIG. 1 is a high-level block diagram of a system for improving quality of patient care according to some embodiments disclosed herein.

FIG. 2A is a high-level block diagram of example procedures that can be used by some embodiments disclosed herein to improve quality of patient care at a medical practice.

FIG. 2B is a high-level block diagram of example procedures that can be used by some embodiments disclosed herein in determining tests, follow ups, and checks ups that are administered to a patient.

FIG. 3 is a high-level block diagram of digital electronic circuitry and hardware that can be used with, incorporated in, or fully or partially included in a system according to the embodiments disclosed herein.

### DETAILED DESCRIPTION

The present disclosure relates to methods, systems, and corresponding apparatus for improving quality of patient care. The disclosed methods, systems, and apparatus can utilize patient information gathered from a variety of sources (e.g., patient health records, electronic health records, insurance records, patient questionnaire records, etc.) to recommend one or more actions for improving quality of care offered to a patient of a medical practice.

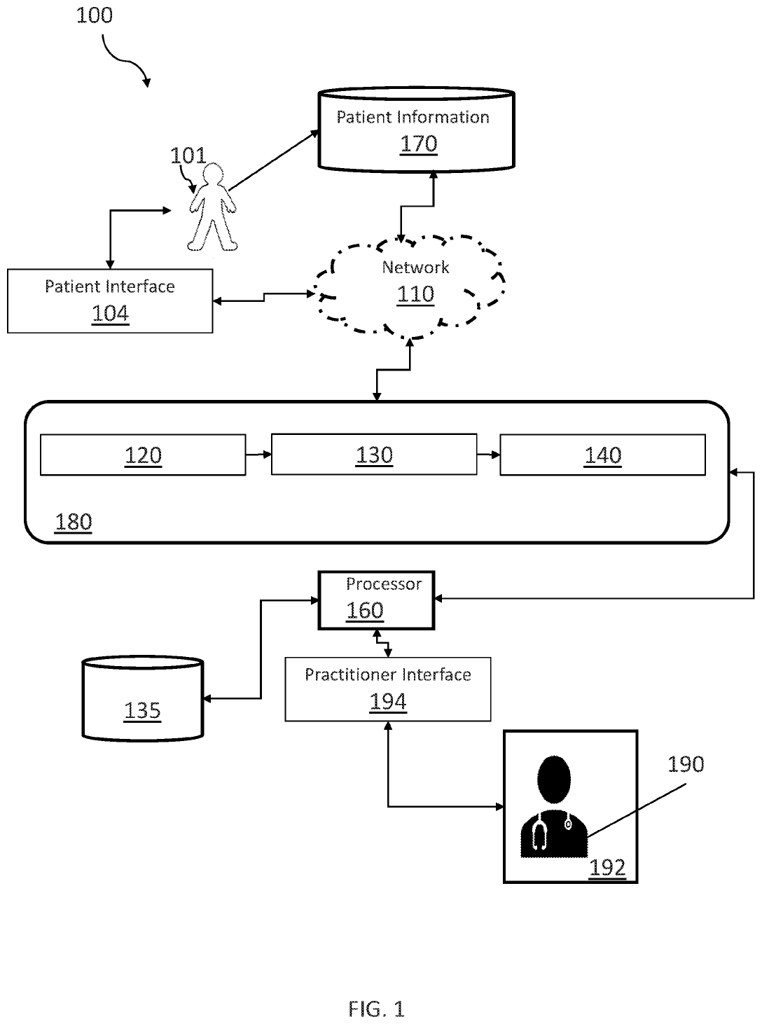

FIG. 1 is a high-level block diagram of a system 100 for improving quality of patient care according to some embodiments disclosed herein. The system 100 can be configured for use at any suitable location 192 that offers medical services to one or more patients. For example, the system 100 can be configured for use in a hospital, a physician's office (e.g., primary care office, ophthalmologist office, etc.) or any other medical office or facility (e.g., pharmacy, optometrist office, physical therapist, etc.) that offers healthcare related services to one or more patients.

The system 100 can comprise a computer-based system that can be hosted/stored in any suitable location. For example, the system 100 can be hosted on a remote server or computer and configured to be accessed via a remote browser or application software on a computer at a medical practice or facility 190. Alternatively or additionally, at least some portion of the system 100 can be hosted on a local computer at the medical practice 190. Generally, the system 100 can be fully or partially hosted/stored in any suitable location that allows access by providers 190 and/or patients 101 via any suitable platform or interface.

The system 100 can comprise one or more databases and information resources 170 that store suitable information regarding a patient 101. The one or more information resources 170 can generally be any suitable resource available in the art. These resources 170 can be internal to the system and/or connected to the system via a suitable communications network 144. For example, the one or more resource 170 can comprise at least one of an electronic health record system (EHR) that stores at least some of a patient's health records, hospital and practice databases (e.g., internal database of a practice, linked databases of specific hospitals and practices that a patient has visited, databases of pharmacies, etc.), insurance records and databases (e.g., database of insurance claims submitted by a medical practice, database of a patient's insurance provider, etc.) and/or any other suitable record system that stores health-related information about the patient.

In addition to the patient's medical data, the one or more resources 170 can store other information and data, such as patient's demographic information, patient's medical history, list of patient's allergies, patient's medications, patient's test results, patient's lab results, patient's imaging results, patient's vital signs and personal statistics (e.g., age, weight, height, temperature, family history, etc.).

The one or more resources 170 can further store any suitable information, such as the patient's insurance records, including prior medical claims submitted to the insurance provider, names and addresses of providers the patient has seen or is scheduled to see, etc. Further, the one or more resources 170 can store insurance billing codes and/or at least some information about the insurance billing codes used to prepare and submit insurance bills for services received by a patient.

The system 100 can further comprise a processor 160 that is configured to implement an analyzer 180 for analyzing patient-specific information included in the one or more resources 170 in order to determine whether the patient-specific information includes at least one identifying feature 120. The at least one identifying feature 120 can be any suitable identifying feature that can be used to classify the patient 101 into at least one class/category for which an action can be recommended.

For example, the at least one identifying feature 120 can be a symptom, condition, or medical or personal history item that is often common among patients having a similar medical condition and/or among patients requiring a similar course of action/treatment. The processor 160 can be configured to use the identifying feature(s) 120 to classify a patient into one or more groups/classes 130 and based on this classification recommend an appropriate course of action and/or treatment plan 140 to the practitioner 192.

The processor 160 can generally be configured to receive the identifying features 120, the corresponding classifications 130, and their appropriate course of action 140 from any suitable source. For example, the processor 160 can obtain this information 120, 130, 140 from a database 135 (e.g., medical database) that stores information on various relevant conditions (i.e., conditions , their common symptoms and identifiers, recommended course of action for addressing those conditions). The database 135 can be specific to the practice 192. For example, in an ophthalmology or an optometry practice, the database can store related to conditions and diseases of the eye and appropriate course of action for addressing such diseases and conditions.

Additionally or alternatively, the processor 160 can be configured to receive at least some of this information from a practitioner and/or a user 190 of the system 100. For example, the system 100 can comprise a practitioner interface 194 that is coupled to the processor 160 and is configured to allow the practitioner 190 to interact with the system 100 and provide the system with customized information. This can allow the practitioner 190 to customize the system 100 to offer recommendations 140 tailored to the specific practice 192, for example based on the equipment available at the practice 192.

For example, the system 100 can be used at a medical practice 192 to create a patient-specific action plan for a patient 101 of a practitioner 190 of the practice 192. The system 100 can access the one or more resources 170 to obtain information regarding the patient 101. As noted above, the one or more resources 170 can include a wide range of databases and resources. For example, the one or more resources 170 can include information provided by the patient to the practice (e.g., in a questionnaire filed by the patient 101 upon arrival) such as patient's age, family history, and reason for visit. The resources 170 can also include information previously collected by the practice 192 (e.g., during patient's previous visits to the practice) such as patient's vitals, patient's medical history as collected and provided by partnering offices and providers (e.g., test results, recent diagnostic images, etc.) , information regarding medications the patient 101 may be taking (e.g., as provided by the patient, the patient's pharmacy, and/or the patient's prescription insurance provider), information obtained from electronic patient systems and electronic billing systems (e.g.,), and/or any other available information from any other available resource.

The one or more resources 170 can also be linked to informational systems and databases that contain additional information regarding the collected medical data. For example, the one or more resources 170 can be linked to a library or database of information that can be used to determine the reasons a patient typically takes a certain medication and/or needed to undergo a certain procedure or test. For example, the information obtained from the resources 170 can indicate that FOSAMAX® is typically administered to patients who suffer from osteoporosis.

The processor 160 can comprise identify one or more preprogrammed identifying features 120 (e.g., family history of breast cancer, family history of diabetes, smoking history, patient BMI, medications a patient takes, history of recent imaging, etc.) and use this information to classify a patient 101 who may have the symptoms or identifier for certain conditions into one or more groups 130 (e.g., group of patients over the age of 40 for screenings that start at 40, group of patients over the age of 50 for screenings that start at 50, group of patients having higher risk of developing breast cancer given family history of breast cancer, group of patients having higher risk of developing diabetes given family history of diabetes, group of patients having higher risk of contracting pneumonia given smoking history, group of patients having higher risk of blood pressure given patient's high BMI, etc.).

Based on these classifications 130, the processor 160 can provide recommendations 140 for tests, procedures, course of action, treatment plans, and/or instructions for each individual patient. As noted, practitioners 190 can customize the recommendations based on resources and equipment available at the practice 192. For example, an ophthalmology practice 192 equipped with an AdaptDx Pro®, manufactured and distributed by MacuLogix, Inc., Harrisburg, Pa., U.S.A., can customize the recommendations offered by the analyzer 180 to recommend using the AdaptDx Pro® to test the patient 190 for macular degeneration (e.g., an early unset of macular degeneration) based on an identifier such as the patient's age (e.g., over 60 years old).

Further, as noted above, the system can include an interactive interface 194 that is configured to receive, from the practitioner 190 or any skilled person, input for use in classifying the practitioners' patients into one or more groups based on certain identifying features. The identifying features 120 can be any suitable features, such as the patient's age, medical history, family history, presence of a new medical condition (e.g., detection of possible diabetes during an ophthalmic test), etc.

The system 100 can use the information to provide the practitioner, his/her colleagues (e.g., other practitioners at that practice), and his/her staff with required information to provide each patient with tailored, patient-specific, medical care. For example, the system 100 can recommend one or more actions, such as tests, medications, follow-ups, etc., for patients belonging to a specific class (e.g., patients belonging to a specific age group, patients having a specific medical history, etc.).

The system can further provide the practitioner, his/her colleagues (e.g., other practitioners at that practice), and his/her staff with additional information and conditions for performing for recommended actions. For example, the patient can indicate the timing and frequency of a specific action (e.g., how often a test should be administered, when the test should be administered (before (in the waiting room), during, or after seeing the physician), and the person responsible for the action (e.g., technician, receptionist, physician, etc.). For example, when in use in a dentist office, the system can review a patient's medical history, including previously used insurance codes for billing for images, to determine whether the patient has had recent images (x-ray) of their teeth. Based on this assessment, the processor 160 can determine whether the patient requires any additional screening (e.g., new x-ray images) and can make recommendations (e.g., regarding taking the new x-ray images) to the practitioner.

As noted the practitioner 190 can customize the recommendations to tailor the needs of his/her practice 192. For example, the practitioner 190 can customize the recommendation such that the system 100 recommends to an assistant of the practitioner 190 to take the required images before the patient 101 is first seen by the practitioner.

Further, in some embodiments, the system can access databases maintaining information on billing practices of a medical facility and/or equipment available at that facility to determine the types of tests and procedures available at that facility. The system can use this information in determining equipment and devices that can be used in testing the patient for certain diseases and conditions. For example, if the system determines that the medical practice owns a dark adaptometer, the system can check the database of patients of that medical to determine whether any of the patients is over a certain age (e.g., sixty) and in need of testing for age-related macular degeneration (e.g., patient has not been recently tested) and in an event a patient meeting such conditions exists (e.g., over sixty and no test in the past three years) recommend testing of the patient using a dark adaptometer.

By providing the recommendations 140, the system can provide uniformity of patient care within a medical practice 192. For example, the system 100 can receive from two or more practitioners (e.g., physicians, ophthalmologists, optometrists, etc.) practicing at a medical practice together information for providing accurate and effective patient care (e.g., tests, follow ups, treatment plans to offer when a patient has a certain condition) and, in response, provide each practitioner at that practice with instructions for providing uniform care to each individual patient of that practice.

As noted, the system 100 can receive information regarding the health histories of all patients of a medical practice (e.g., information such as the patient's age, medical history, family history, presence of a new medical condition), classify the patients based on their health histories, and recommend, to all practitioners at that practice, uniform actions for all patients falling within similar classes. For example, the system can monitor all patients of the practice and determine whether the patient is at least 60 years old and also whether the patient has been tested for age-related macular degeneration (AMD) within the past three years. If the patient satisfies both conditions (over sixty and no test in the past three years), the system can prompt a technician at the facility to conduct a test directed to detection of AMD on the patient and provide the physician with the results before the patient is seen by the physician. This ensures that all patients to the medical facility receive uniform care, regardless of which physician at the facility they are seeing.

The system 100 can also be utilized in providing continuity of care to the patients of a practice. Specifically, as shown in FIG. 1, the system 100 can comprise a patient interface 104 that is configured to interact with the patient and receive data and information from the patient and/or present the patient with data and information. The patient interface 104 can be an integral part of the system 100 and/or connected to the system via a suitable means of communication, for example through a communications network 104. In some embodiments, the patient interface can be a web-based and/or application software based system that is used by the patient to interact with the system 100. Generally, any suitable platform can be used to communicate with the patient 101. For example, in some implementations, the system 100 can be configured to communicate with the patient 101 via text messages, phone calls, and/or emails.

In general, the system 100 can receive, from a care provider (e.g., a physician, an ophthalmologist, an optometrist, etc.) information for providing accurate and effective patient care and, in response, provide patients of that provider, and/or a medical practice to which the provider belongs, with appropriate patient-specific continuity of care information via the patient interface 104.

For example, the system 100 can provide a patient 101, who has been determined to need certain medications with updates regarding taking his/her medication and/or with requests for confirmation of whether the patient has taken their medication (e.g., via a text message asking the patient if they have taken their medication at a predetermined time and requesting a response (Y/N)).

Further, the system 100 can assist a patient in monitoring his/her medical condition by receiving, from the patient, a measure indicating progress of a disease or condition. For example, the patient 101 can be a patient who is recovering at home after a total knee replacement surgery. The system 100 can classify the patient 101 into a classification group 130 of patient requiring monitoring while recovering at home and recommend certain screening factors 140 for the patient (e.g., has the patient moved and used the replaced knee?, does the patient have a fever that can indicate a possible infection? Is the wound healing or if there is any drainage?). The processor 160 can monitor the progress of those screening factors 140 through the patient interface 104 and require the patient 101 to provide a response (e.g., yes, I have walked around, no fever, some swelling and redness around the incision area). The processor can process this information using the identification 120, classification 130, and recommendation 140 schemes described above, and determine if the patient's condition needs to be brought to the practitioner's attention. For example, the system 100 can determine the condition of similar patients (e.g., similar age group, body type, weight, gender, etc.) who have gone through the same course of treatment (e.g., patients who are three days post a total knee replacement operation, patients who are being treated for diabetes, patients who recently had laser eye surgery, etc.) and determine whether the patient's response requires physician's attention (e.g., by classifying the patient in a group whose symptoms fall outside of the normal or expected range (e.g., patients are not expected to have a fever or experience significant swelling, redness, or drainage around the incision area. Therefore, a patient indicating presence of drainage, redness, or swelling is outside the expected range). Based on this information, the processor 160 can provide the practitioner 190 with a recommendation for following up with the patient 101. Alternatively or additionally, the system 100 can prompt the patient to follow up with the practitioner 190.

Therefore, by evaluating the progress of a condition, and in response to detection of an undesired progress, the system 100 can prompt the patient to seek care and/or adjust the care provided to the patient. Further, in some implementations, the system can comprise an interface that receives from the patient information regarding various test results (e.g., home blood pressure monitoring, home blood sugar monitoring, tests obtained at various medical facilities, for example two or more dark adaptation tests obtained using a dark adaptometer over a period of time) and determine if the patient's condition has progressed/changed and based on that determination recommends a follow up with the practitioner to the patient.

Generally, the user interface 104 and/or the physician interface 194 can comprise any suitable interface that is configured to receive an input 150 from a user 101/190. For example, the user interface 104/190 can comprise at least one of: a display (e.g., an interactive display), a keyboard, a touch pad, a button or a computer mouse, an audio speaker, an audio microphone, etc.

Further, although described as a medical practitioner, the user 190 can be any suitable skilled person such as a care provider (e.g., a physician, an ophthalmologist, an optometrist, etc.). The user 190 can use the interface 194 to present information 150 for providing accurate, consistent, uniform, and effective patient care to the system 100. As detailed above, the system 100 can comprise a processor 160 that is configured to receive the information provided by the user 190 and store the information in a database 135 for further processing.

For example, the user can be a practitioner (e.g., a physician or an ophthalmologist) that provides the system with certain information 150 regarding tests, follow ups, and checks ups that should be provided to a specific group of patients (e.g., patients over the age of 60) that seek care at the provider's ophthalmology practice. For example, an ophthalmologist 190 can use the system 100 to ensure that all patients over the age of 60 who visit her practice, and have not been tested for markers indicating an early onset of age-related macular degeneration, receive appropriate tests to determine whether they exhibit signs and/or symptoms of this disease.

As noted, the user 190 can further provide the system with one or more actions 140 to be conducted for each patient determined to belong to one or more specified groups. For example, the user 190 can provide the system with one or more tests, procedures, and/or actions to be conducted. The tests, procedures, and actions can be any suitable tests, procedures, or actions. For example, the tests, procedures, and actions can be directed at the detection and diagnosis of various ophthalmic conditions and diseases, such as age-related macular degeneration, vitamin A deficiency, Sorsby's Fundus Dystrophy, late autosomal dominant retinal degeneration, retinal impairment related to diabetes, diabetic retinopathy, retinitis pigmentosa, visual field tests directed to detecting dysfunctions in the central and peripheral vision, which may be caused by medical conditions such as glaucoma, pituitary diseases, strokes, brain tumors, or other neurological issues.

The user 190 can further provide the system with additional information and instructions regarding each of the one or more actions 160. The information and instructions can include any suitable information and instructions. For example, the user 190 can provide the system with information 170 regarding whether certain tests should be administered before or after the patient is seen by a physician, whether the patient should be scheduled for a follow up visit, when the patient should return for his/her follow up visit, whether the patient should be provided with a referral to a different practitioner, etc.

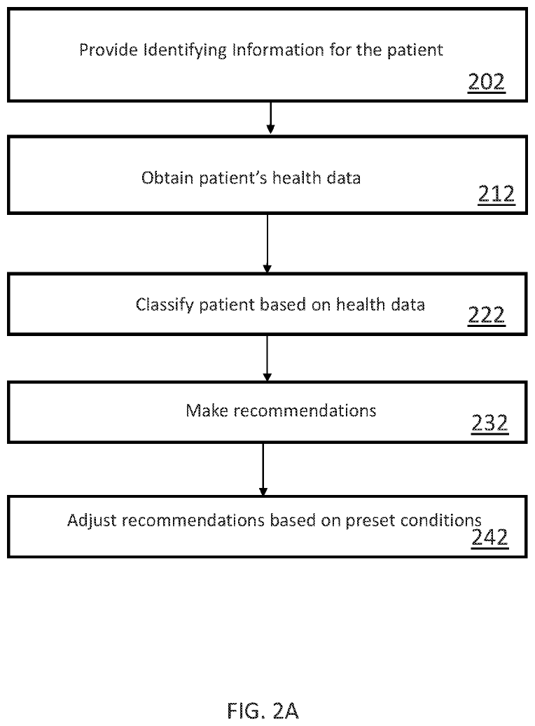

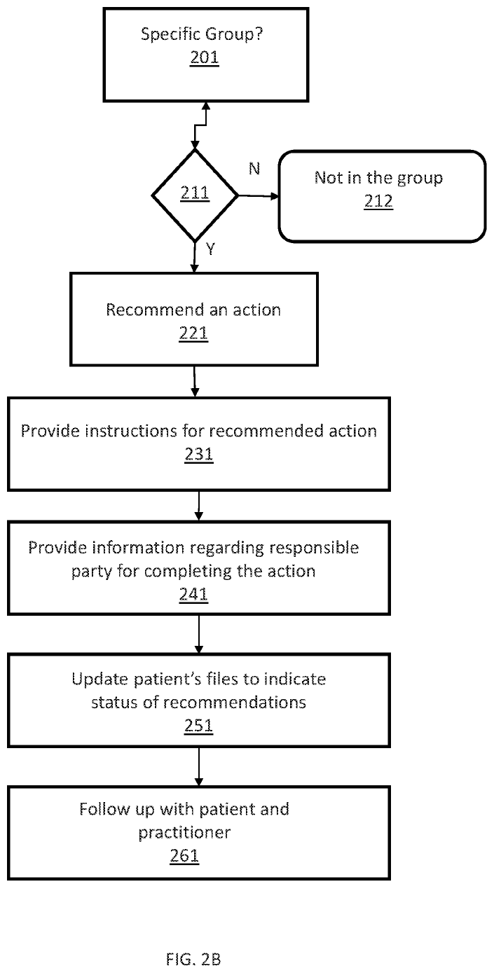

FIG. 2A is a high-level block diagram of example procedures that can be used by some embodiments disclosed herein to improve quality of patient care at a medical practice. As shown in FIG. 2A, a user can initiate a system for improving quality of patient care 100 by providing one or more conditions for classifying and identifying the patients visiting his/her practice 202. For example, the user can provide the system with information regarding patients who are scheduled to visit her practice on a certain day 202. In response, the system obtains the patients' health information 212 and classifies the patients based on their age and medical history (e.g., pre-existing conditions, family history, whether they have been previously tested for a condition or disease, whether they are due for a test, etc.) 222. In some implementations, the system 100 can be configured to obtain the information regarding the patients visiting the practice from the physician's scheduling system or tool.

The user can also provide the system with information regarding one or more actions for performing on each individual patient based on determining whether the patient belongs to a certain group of patients. Based on this information and the classification information, the system can recommend one or more actions for performing on the patient 232 (e.g., test a patient who is at least sixty years old and has not been tested in the past three years for onset of AMD).

As noted, the user can also provide the system with one or more conditions for performing the actions and the system can adjust its recommendations based on these conditions 232. The conditions can be any suitable action. For example, the conditions can include whether the actions to be performed should be performed before or after the patient is first seen by the physician (e.g., patient can be tested for AMD before she/he sees the ophthalmologist such that the results of the test are available to ophthalmologist as soon as he/she first encounters the patient). However, if the patient's eyes are being dilated, this should be done after the patient first encounters the ophthalmologist. The conditions can also clarify the party responsible for taking the actions to be performed. For example, if the action comprises obtaining dark adaptation measurements, the action can be performed by a technician, whereas if the action comprises requiring a patient to come back for a follow-up visit, a scheduling system can be triggered to provide the patient with a follow up appointment.

FIG. 2B is a high-level block diagram of example procedures that can be used by some embodiments disclosed herein in determining tests, follow ups, and checks ups that are administered to a patient. As noted with respect to FIG. 1, the system can determine whether a patient belongs to a certain/specified group of patients 201. For example, the system can determine 211 whether the patient is over a certain age (e.g., patient is at least 60 years old, patient is at or near an age where onset of one or more diseases is typically detected), patient is due for certain tests (e.g., patient has not had her retina checked in the past two years and is due for a screening), and/or has family history of a certain disease (e.g., family history of retinitis pigmentosa).

If the system determines that the patient does not belong to the specified group (N), the system can generate a notification 212 that the patient does not belong to the specified group and/or simply record in the database that the association of the patient with the specified group has been verified and the patient is determined not to belong to the specified group. Alternatively or additionally, the system can proceed with verifying the patient's association with other groups (e.g., determine that patient is under a specified age range but is due for a certain procedure and/or has family history of a certain disease or condition).

If the patient is determined to belong to the specified group (Y), the system can proceed with providing instructions for administering one or more tests, procedures, and actions on the patient 221. As noted above, the procedures can include any suitable test, procedure, and/or actions. For example, the system can proceed with providing instructions for performing one or more of dark adaptation measurement, Fundus Retinal Imaging, Retinal Densitometry, Optical Coherence Tomography (OCT), Fluorescein Angiography, OCT Angiography (OCTA), Multi-spectral Imaging, Scanning Laser Ophthalmoscope, Anterior Segment OCT, Deep-field OCT, Retinal Metabolic Imaging, Ocular Blood Flow Imaging, Adaptive Optics, Autofluorescence, Non-mydriatic Fundus Camera, Optic Nerve Imaging, Ultrasound, Anterior Segment Photography, Slit Lamp, and Refractive Eye Care testing including functions of a Pachymeter and Interior Segment testing functions.

Further, the system can determine whether any additional information or instructions regarding a recommended test, procedure, and action is available 231. The additional instructions can be any suitable information. For example, the additional instructions can include information regarding whether a test should be conducted before or after the patient has first encounter a physician. The system can also specify a responsible party for conducting a recommended test, procedure, and action 241. For example, depending on the action being performed, the system can specify whether a technician, an office staff, a care provider, or a computer-based system can conduct the recommended action.

The system can also update the patient file to indicate the status of the recommended test, procedure, and action 251 and conduct any subsequent action required for monitoring the status 261. For example, if the recommended action comprises administration of certain drug (e.g., requiring that a smoker patient takes a certain medication to assist with quitting smoking), the system can follow up 261 with the patient (e.g., via automated text messages) to inquire whether the patient is taking such medications. In some embodiments, the system can require the patient to provide responses to such inquiries (e.g., confirm that the patient has taken the medication, confirm that the patient has gone for the follow up visit, etc.) and update the patient's chart accordingly.

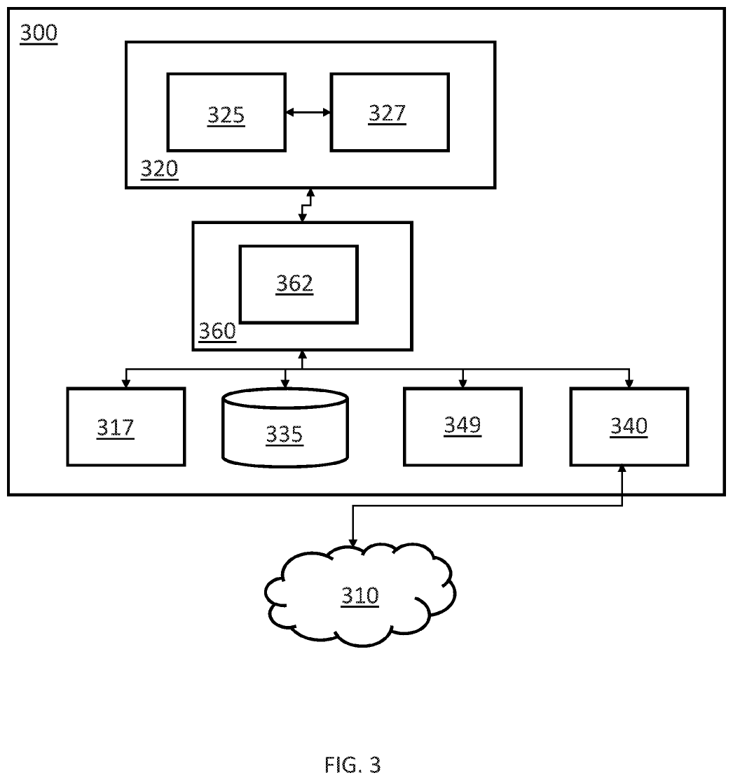

FIG. 3 is a high-level block diagram of digital electronic circuitry and hardware 300 that can be used with, incorporated in, or fully or partially included in a system according to the embodiments disclosed herein. The electric circuitry 300 can include a processor 360 that is configured to monitor the system, send and/or receive signals for operating the system, and/or control the operation of the system.

The processor 360 can be configured to collect or receive information and data regarding the operation of the system 100 and/or store or forward information and data to another entity. For example, the processor 360 can be configured to access billing and insurance data of a specific patient in order to determine whether a patient has received a test previously, whether appropriate authorizations for conducting a test are in place and/or should be obtained.

The processor 360 can further be configured to control, monitor, and/or carry out various functions needed for analysis, interpretation, tracking, and reporting of information and data collected by the system 100. Generally, these functions can be carried out and implemented by any suitable computer system and/or in digital circuitry or computer hardware, and the processor 310 can implement and/or control the various functions and methods described herein.

The processor 360 can be connected to a main memory 320, and comprise a central processing unit (CPU) 362 that includes processing circuitry configured to manipulate instructions received from the main memory 320 and execute various instructions. The CPU 360 can be any suitable processing unit known in the art. For example, the CPU 360 can be a general and/or special purpose microprocessor, such as an application-specific instruction set processor, graphics processing unit, physics processing unit, digital signal processor, image processor, coprocessor, floating-point processor, network processor, and/or any other suitable processor that can be used in a digital computing circuitry. Alternatively or additionally, the processor can comprise at least one of a multi-core processor and a front-end processor.

Generally, the processor 360 and the CPU 362 can be configured to receive instructions and data from the main memory 320 (e.g., a read-only memory or a random access memory or both) and execute the instructions. The instructions and other data can be stored in the main memory 320. The processor 360 and the main memory 320 can be included in or supplemented by special purpose logic circuitry. The main memory 320 can be any suitable form of volatile memory, non-volatile memory, semi-volatile memory, or virtual memory included in machine-readable storage devices suitable for embodying data and computer program instructions. For example, the main memory 320 can comprise magnetic disks (e.g., internal or removable disks), magneto-optical disks, one or more of a semiconductor memory device (e.g., EPROM or EEPROM), flash memory, CD-ROM, and/or DVD-ROM disks.

The main memory 320 can comprise an operating system 325 that is configured to implement various operating system functions. For example, the operating system 325 can be responsible for controlling access to various devices, memory management, and/or implementing various functions of the system 100. Generally, the operating system 325 can be any suitable system software that can manage computer hardware and software resources and provide common services for computer programs.

The main memory 320 can also hold application software 327. For example, the main memory 320 and application software 327 can include various computer executable instructions, application software, and data structures, such as computer executable instructions and data structures that implement various aspects of the embodiments described herein. For example, the main memory 320 and application software 327 can include computer executable instructions, application software, and data structures, such as computer executable instructions and data structures that implement the system for improving the quality of patient care.

Generally, the functions performed by system 100 can be implemented in digital electronic circuitry or in computer hardware that executes software, firmware, or combinations thereof. The implementation can be as a computer program product (e.g., a computer program tangibly embodied in a non-transitory machine-readable storage device) for execution by or to control the operation of a data processing apparatus (e.g., a computer, a programmable processor, or multiple computers).

The main memory 320 can also be connected to a cache unit (not shown) configured to store copies of the data from the most frequently used main memory 320. The program codes that can be used with the embodiments disclosed herein can be implemented and written in any form of programming language, including compiled or interpreted languages, and can be deployed in any form, including as a stand-alone program or as a component, module, subroutine, or other unit suitable for use in a computing environment. A computer program can be configured to be executed on a computer, or on multiple computers, at one site or distributed across multiple sites and interconnected by a communications network, such as the Internet.

The processor 360 can further be coupled to a database or data storage 330. The data storage 335 can be configured to store information and data relating to various functions and operations of the ophthalmic testing and measurement system 150. For example, the data storage 335 can store the data collected by the system 100.

The processor 360 can further be coupled to a display 317. The display 317 can be configured to receive information and instructions from the processor. The display 317 can generally be any suitable display available in the art, for example a Liquid Crystal Display (LCD) or a light emitting diode (LED) display. For example, the display 317 can be a smart and/or touch sensitive display that can receive instructions from a user and/or provide information to the user.

The processor 360 can further be connected to various interfaces, such as a user's interface. The connection to the various interfaces can be established via a system or an input/output (I/O) interface 394 (e.g., Bluetooth, USB connector, audio interface, FireWire, interface for connecting peripheral devices, etc.). The I/O interface 394 can be directly or indirectly connected to the system 100.

The processor 360 can further be coupled to a communication interface 340, such as a network interface. The communication interface 340 can be a communication interface that is included in the system 100 and/or a remote communications interface 340 that is configured to communicate with the system 100. For example, the communications interface 340 can be a communications interface that is configured to provide the system 100 with a connection to a suitable communications network 310, such as the Internet. Transmission and reception of data, information, and instructions can occur over the communications network 310. Further, in some embodiments, the communications interface 340 can be an interface that is configured to allow communication between the digital circuitry 300 (e.g., a remote computer) and the system 100 (e.g., via any suitable communications means such as a wired or wireless communications protocols including WIFI and Bluetooth communications schemes).

Those having ordinary skill in the art will appreciate that various changes can be made to the above embodiments without departing from the scope of the invention. Although this specification discloses advantages in the context of certain illustrative, non-limiting embodiments, various changes, substitutions, permutations, and alterations may be made without departing from the scope of the specification as defined by the appended claims. Further, any feature described in connection with any one embodiment may also be applicable to any other embodiment.

What is claimed is:

1. A system for improving quality of healthcare service provided to a patient of a medical practice, the system comprising:
one or more processors;
a memory coupled to the one or more processors; and
one or more programs stored in the memory and configured to be executed by the one or more processors, the one or more programs including instructions that upon execution:
receive information identifying the patient of the medical practice;
classify the patient into one or more categories based on at least one predetermined criterion;
recommend at least one healthcare service action for providing to the patient based on the one or more categories; and
provide a practitioner at the medical practice with information relating to the at least one healthcare service action.

2. The system of claim 1, wherein the information identifying the patient comprises information stored on at least one electronic health record system that stores at least some information about the patient.

3. The system of claim 1, wherein the information identifying the patient comprises information stored on an internal database of the medical practice.

4. The system of claim 1, wherein the information identifying the patient comprises information relating to at least one of patient's health records, patient's insurance records, patient's biographic records, patient's demographic records, and patient's insurance billing records.

5. The system of claim 1, further comprising an interactive practitioner interface coupled to the processor and configured to provide the practitioner with the at least one healthcare service action.

6. The system of claim 5, wherein the one or more programs further include instructions that upon execution receive the at least one criterion from the practitioner via the interactive practitioner interface.

7. The system of claim 1, wherein the at least one criterion is at least one of a feature, a symptom, a condition, a medical history item, or a personal history item common to patients requiring the at least one healthcare service action.

8. The system of claim 1, wherein the one or more programs include instructions that upon execution obtain information relating to the at least one criterion from the information identifying the patient.

9. The system of claim 1, wherein the at least one healthcare service action comprises a follow up action with the patient.

10. The system of claim 1, further comprising an interactive patient interface coupled to the processor and configured to receive at least some of information identifying the patient from the patient.

11. The system of claim 1, further comprising an interactive patient interface coupled to the processor and configured to provide the patient with at least some information relating to the at least one healthcare service item.

12. The system of claim 11, wherein the interactive patient interface is further configured to receive a response from the patient in connection with the at least one healthcare service item.

13. The system of claim 12, wherein the one or more programs further include instructions that upon execution analyze the response received from the patient and classify the response into the one or more updated categories based on the at least one predetermined criterion.

14. The system of claim 13, wherein the one or more programs further include instructions that upon execution recommend at least one updated healthcare service action based on the one or more updated categories.
